(19) United States
(12) Patent Application Publication
(10) Pub. No.: US 2023/0390733 A1
(43) Pub. Date: Dec. 7, 2023

(54) METHODS AND SYSTEMS FOR ISOMERIC SEPARATION USING MESOPOROUS GRAPHITIZED CARBON

(71) Applicants: Yehia MCHREF, Lubbock, TX (US); Alireza BANAZADEH, Lubbock, TX (US); Sakshi GAUTAM, Lubbock, TX (US); Byeong Gwan CHO, Lubbock, TX (US)

(72) Inventors: Yehia Mechref, Lubbock, TX (US); Alireza Banazadeh, Lubbock, TX (US); Sakshi Gautam, Lubbock, TX (US); Byeong Gwan Cho, Lubbock, TX (US); Mona Goli, Lubbock, TX (US)

(21) Appl. No.: 18/250,892

(22) PCT Filed: Oct. 27, 2021
(86) PCT No.: PCT/US2021/056894

§ 371 Date: Apr. 27, 2023

Related U.S. Application Data
(60) Provisional application No. 63/106,277, filed on Oct. 27, 2020.

(51) Int. Cl. B01J 20/20; B01D 15/20; B01D 15/22; C07K 1/22; B01J 20/28; B01J 20/282

(52) U.S. Cl. CPC B01J 20/20; B01D 15/206; B01D 15/22; C07K 1/22; B01J 20/28083; B01J 20/282; B01J 2220/58

(57) ABSTRACT

A method, system, and apparatus for chromatography comprises a chromatographic system can include a column packed with mesoporous graphitized carbon (MGC). The MGC serves as an ideal stationary phase and facilitates the efficient isomeric separation of compounds such as permethylated glycans with unprecedented 10 mm long column. The column is anticipated to be an efficient and economical replacement for nanoflow PGC column which was phased out by the manufacturer due to poor performance and lack of reproducibility.

### CROSS REFERENCE TO RELATED PATENT APPLICATIONS

This application claims the priority and benefit under 35 U.S.C. § 119(e) of U.S. Provisional Patent Application Ser. No. 63/106,277 filed Sep. 27, 2020, entitled “METHODS AND SYSTEMS FOR ISOMERIC SEPARATION USING MESOPOROUS GRAPHITIZED CARBON.” U.S. Provisional Patent Application Ser. No. 63/106,277 is herein incorporated by reference in its entirety.

### STATEMENT OF GOVERNMENT RIGHTS

The invention described in this patent application was made with Government support under the projects titled “Sensitive and Quantitative MS-Based Gloycomic Mapping Platform” and “Quantitative Characterization of Glycopeptide Isomers”, Contract Numbers 501GM11249006 and 1R01GM13009101A1 respectively, awarded by the National Institutes of Health. The Government may have certain rights in the invention.

### TECHNICAL FIELD

Embodiments are generally related to the field of chromatographic devices. Embodiments are also related to isomeric separation systems and methods. Embodiments are further related to the use of Mesoporous Graphitized Carbon used in chromatographic devices. Embodiments are further related to methods and systems for efficient isomeric separation of glycans and glycopeptides using Mesoporous Graphitized Carbon packed columns.

### BACKGROUND

Chromatography is a well-known and widely used analytical technique. Chromatographic techniques facilitate separation of a mixture of chemical compounds into constituent components, so that one or more of the constituent components can be analyzed individually. There are various techniques for chromatography, but most of the known techniques and devices suffer from various drawbacks. For example, many such techniques and devices are prohibitively expensive, or lack robustness, and fail to provide consistent performance as necessary for biological or chemical testing.

Glycans, which are composed of single sugar molecules in a chain-like structure, are important for several biological processes such as cell signaling, protein stability, immune response, and host-pathogen interactions. Glycosylation is one of the most important post-translational modifications of proteins. Extensive heterogeneity of the glycans complicates their investigation in biological systems. The different isomeric forms of glycans demonstrate different biological functions in organisms. Researchers have found associations with diseases such as breast cancer, pancreatic cancer, and Alzheimer's and monosaccharaide residues with specific linkages. Moreover, different linkage-based glycan isomers have also been demonstrated to be responsible for host-pathogen interactions, like influenza virus infection. As such, isomeric separation and identification is critically important in medical applications. Reliable isomeric glycan quantitation will allow for a better understanding of glycan biofunctions and their relationships to disease, thereby providing reliable scientific data for the development of efficient vaccines and therapeutics.

A widely used glycan derivatization method is permethylation, which has advantages of enhancing a mass spectrometry signal and stabilizing glycan structures by preventing sialic acid loss and fucose migration. This chemical alteration enhances ionization capabilities as well as molecular stability of glycans in the gas phase.

Glycans have been separated using Hydrophilic Interaction Liquid Chromatography (HILIC) and Porous Graphitized Carbon (PGC) columns to achieve good peak shape and high isomeric resolution. PGC is an important chromatographic tool because it retains polar compounds with mass spectrometry-compatible solvents. Due to its hydrophobic and polar interactions with glycan species, PGC is the stationary phase of choice in prior art approaches to nano-Liquid Chromatography (nano-LC) based isomeric separation of permethylated glycans. Such PGC columns commonly have a length of 100 mm and are packed with particles 5 μm in size.

PGC columns can be used to separate hydrophilic analytes and permethylated glycans, but PGC columns also include numerous limitations. For example, PGC columns offer poor reproducibility and require regeneration after even a few sample runs. Prior art approaches to chromatography with PGC have, at present, failed to ameliorate this problem. As a result, PGC column manufacturers have effectively phased out associated product lines. This has led to a critical need for testing equipment capable of filling the void.

Accordingly, there is a need for systems and methods that can be used to provide efficient, cost effective, and reliable chromatography, particularly for efficient isomeric separation of glycans and glycopeptides, as disclosed herein.

### SUMMARY

The following summary is provided to facilitate an understanding of some of the innovative features unique to the embodiments disclosed and is not intended to be a full description. A full appreciation of the various aspects of the embodiments can be gained by taking the entire specification, claims, drawings, and abstract as a whole.

It is, therefore, one aspect of the disclosed embodiments to provide chromatography systems and methods.

It is another aspect of the disclosed embodiments to provide techniques for separation of glycans and glycopeptides.

It is another aspect of the disclosed embodiments to provide method Mesoporous Graphitized Carbon as a stationary phase for chromatography devices.

It is another aspect of the disclosed embodiments to provide methods and systems for isomeric separation of glycans and glycopeptides using Mesoporous Graphitized Carbon packed chromatographs.

It will be appreciated that the methods and systems can be achieved according to the embodiments disclosed herein. For example, in an embodiment, a chromatography system comprises a column comprising a pulled capillary nanospray emitter column, a stationary phase comprising mesoporous graphitized carbon, frit inserted in the column and a heater configured for controlling a temperature of the pulled capillary nanospray emitter column. In an embodiment, the mesoporous graphitized carbon is selected to have a size of less than 500 nm and a pore size of 64 Angstroms, and the mesoporous graphitized carbon in a packed into 10 mm or less of the pulled capillary nanospray emitter column.

### BRIEF DESCRIPTION OF THE FIGURES

The accompanying figures, in which like reference numerals refer to identical or functionally-similar elements throughout the separate views and which are incorporated in and form a part of the specification, further illustrate the embodiments and, together with the detailed description, serve to explain the embodiments disclosed herein.

FIG. 1 depicts a block diagram of a computer system which is implemented in accordance with the disclosed embodiments;

FIG. 2 depicts a graphical representation of a network of data-processing devices in which aspects of the present embodiments may be implemented;

FIG. 3 depicts a computer software system for directing the operation of the data-processing system depicted in FIG. 1, in accordance with an example embodiment;

FIG. 4 depicts an illustration of MGC particles, in accordance with the disclosed embodiments;

FIG. 5A depicts a chromatography system, in accordance with the disclosed embodiments;

FIG. 5B depicts a column packing assembly, in accordance with the disclosed embodiments;

FIG. 6 depicts a chart of sample results, in accordance with the disclosed embodiments;

FIG. 7A depicts a chart of sample results, in accordance with the disclosed embodiments;

FIG. 7B depicts charts illustrating separation of isomeric permethylated glycans derived from MDA-MB231, MDA-MB-231BR and CRL-1620 with an MGC column, in accordance with the disclosed embodiments;

FIG. 8 depicts a flow chart of steps associated with a chromatography method, in accordance with the disclosed embodiments;

FIG. 9 depicts a block diagram of a heater system, in accordance with the disclosed embodiments;

FIG. 10 depicts comparative results of the system with and without use of the heater, in accordance with the disclosed embodiments;

FIG. 11 depicts a needle array assembly, in accordance with the disclosed embodiments;

FIG. 12 illustrates an MS/MS spectrum of isomeric A2G2S2 glycopeptide from bovine fetuin separated on an MGC column in accordance with the disclosed embodiments; and

FIG. 13 illustrates a chromatogram showing the isomeric separation of glycopeptides derived from alpha-1 acid glycoprotein on an MGC column, in accordance with the disclosed embodiments.

### DETAILED DESCRIPTION

The particular values and configurations discussed in the following non-limiting examples can be varied, and are cited merely to illustrate one or more embodiments, and are not intended to limit the scope thereof.

Example embodiments will now be described more fully hereinafter, with reference to the accompanying drawings, in which illustrative embodiments are shown. The embodiments disclosed herein can be embodied in many different forms and should not be construed as limited to the embodiments set forth herein; rather, these embodiments are provided so that this disclosure will be thorough and complete, and will fully convey the scope of the embodiments to those skilled in the art. Like numbers refer to like elements throughout.

The terminology used herein is for the purpose of describing particular embodiments only and is not intended to be limiting. As used herein, the singular forms “a”, “an”, and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Throughout the specification and claims, terms may have nuanced meanings suggested or implied in context beyond an explicitly stated meaning. Likewise, the phrase “in one embodiment” as used herein does not necessarily refer to the same embodiment and the phrase “in another embodiment” as used herein does not necessarily refer to a different embodiment. It is intended, for example, that claimed subject matter include combinations of example embodiments in whole or in part.

Unless otherwise defined, all terms (including technical and scientific terms) used herein have the same meaning as commonly understood by one of ordinary skill in the art. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit, and scope of the invention. All such similar substitutes and modifications apparent to those skilled in the art are deemed to be within the spirit, scope and concept of the invention as defined by the appended claims.

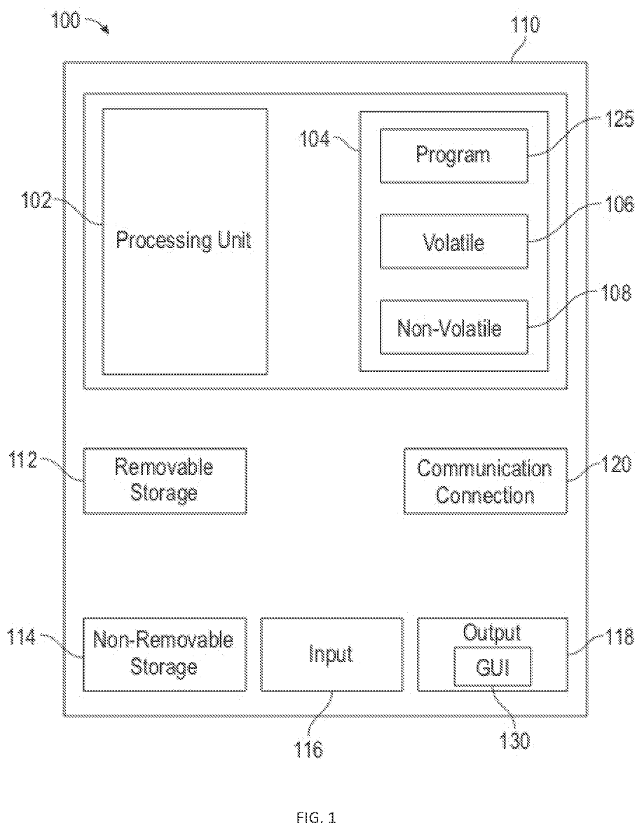

FIGS. 1-3 are provided as exemplary diagrams of data-processing environments in which embodiments of the present embodiments may be implemented. It should be appreciated that FIGS. 1-3 are only exemplary and are not intended to assert or imply any limitation with regard to the environments in which aspects or embodiments of the disclosed embodiments may be implemented. Many modifications to the depicted environments may be made without departing from the spirit and scope of the disclosed embodiments.

A block diagram of a computer system 100 that executes programming for implementing parts of the methods and systems disclosed herein is shown in FIG. 1. A computing device in the form of a computer 110 configured to interface with controllers, peripheral devices, and other elements disclosed herein may include one or more processing units 102, memory 104, removable storage 112, and non-removable storage 114. Memory 104 may include volatile memory 106 and non-volatile memory 108. Computer 110 may include or have access to a computing environment that includes a variety of transitory and non-transitory computer-readable media such as volatile memory 106 and non-volatile memory 108, removable storage 112 and non-removable storage 114. Computer storage includes, for example, random access memory (RAM), read only memory (ROM), erasable programmable read-only memory (EPROM) and electrically erasable programmable read-only memory (EEPROM), flash memory or other memory technologies, compact disc read-only memory (CD ROM), Digital Versatile Disks (DVD) or other optical disk storage, magnetic cassettes, magnetic tape, magnetic disk storage, or other magnetic storage devices, or any other medium capable of storing computer-readable instructions as well as data including image data.

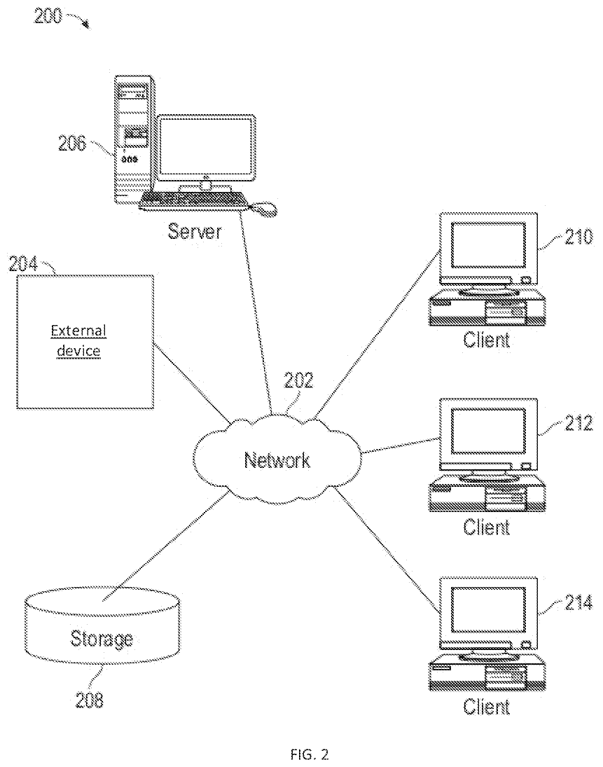

Computer 110 may include, or have access to, a computing environment that includes input 116, output 118, and a communication connection 120. The computer may operate in a networked environment using a communication connection 120 to connect to one or more remote computers, remote sensors and/or controllers, detection devices, hand-held devices, multi-function devices (MFDs), speakers, mobile devices, tablet devices, mobile phones, Smartphone, or other such devices. The remote computer may also include a personal computer (PC), server, router, network PC, RFID enabled device, a peer device or other common network node, or the like. The communication connection may include a Local Area Network (LAN), a Wide Area Network (WAN), Bluetooth connection, or other networks. This functionality is described more fully in the description associated with FIG. 2 below.

Output 118 is most commonly provided as a computer monitor, but may include any output device. Output 118 and/or input 116 may include a data collection apparatus associated with computer system 100. In addition, input 116, which commonly includes a computer keyboard and/or pointing device such as a computer mouse, computer track pad, or the like, allows a user to select and instruct computer system 100. A user interface can be provided using output 118 and input 116. Output 118 may function as a display for displaying data and information for a user, and for interactively displaying a graphical user interface (GUI) 130.

Note that the term “GUI” generally refers to a type of environment that represents programs, files, options, and so forth by means of graphically displayed icons, menus, and dialog boxes on a computer monitor screen. A user can interact with the GUI to select and activate such options by directly touching the screen and/or pointing and clicking with a user input device 116 such as, for example, a pointing device such as a mouse, and/or with a keyboard. A particular item can function in the same manner to the user in all applications because the GUI provides standard software routines (e.g., module 125) to handle these elements and report the user's actions. The GUI can further be used to display the electronic service image frames as discussed below.

Computer-readable instructions, for example, program module or node 125, which can be representative of other modules or nodes described herein, are stored on a computer-readable medium and are executable by the processing unit 102 of computer 110. Program module or node 125 may include a computer application. A hard drive, CD-ROM, RAM, Flash Memory, and a USB drive are just some examples of articles including a computer-readable medium.

FIG. 2 depicts a graphical representation of a network of data-processing systems 200 in which aspects of the present invention may be implemented. Network data-processing system 200 can be a network of computers or other such devices, such as mobile phones, smart phones, sensors, controllers, actuators, speakers, “internet of things” devices, and the like, in which embodiments of the present invention may be implemented. Note that the system 200 can be implemented in the context of a software module such as program module 125. The system 200 includes a network 202 in communication with one or more clients 210, 212, and 214. Network 202 may also be in communication with one or more devices 204, servers 206, and storage 208. Network 202 is a medium that can be used to provide communications links between various devices and computers connected together within a networked data processing system such as computer system 100. Network 202 may include connections such as wired communication links, wireless communication links of various types, and fiber optic cables. Network 202 can communicate with one or more servers 206, one or more external devices such as device 204, and a memory storage unit such as, for example, memory or database 208. It should be understood that device 204 may be embodied as a detector device, controller, receiver, transmitter, transceiver, transducer, driver, signal generator, some combination thereof, or other such device.

In the depicted example, device 204, server 206, and clients 210, 212, and 214 connect to network 202 along with storage unit 208. Clients 210, 212, and 214 may be, for example, personal computers or network computers, handheld devices, mobile devices, tablet devices, smart phones, personal digital assistants, controllers, recording devices, speakers, MFDs, etc. Computer system 100 depicted in FIG. 1 can be, for example, a client such as client 210 and/or 212 and/or 214.

Computer system 100 can also be implemented as a server such as server 206, depending upon design considerations. In the depicted example, server 206 provides data such as boot files, operating system images, applications, and application updates to clients 210, 212, and/or 214. Clients 210, 212, and 214 and device 204 are clients to server 206 in this example. Network data-processing system 200 may include additional servers, clients, and other devices not shown. Specifically, clients may connect to any member of a network of servers, which provide equivalent content.

In the depicted example, network data-processing system 200 is the Internet, with network 202 representing a worldwide collection of networks and gateways that use the Transmission Control Protocol/Internet Protocol (TCP/IP) suite of protocols to communicate with one another. At the heart of the Internet is a backbone of high-speed data communication lines between major nodes or host computers consisting of thousands of commercial, government, educational, and other computer systems that route data and messages. Of course, network data-processing system 200 may also be implemented as a number of different types of networks such as, for example, an intranet, a local area network (LAN), or a wide area network (WAN). FIGS. 1 and 2 are intended as examples and not as architectural limitations for different embodiments of the present invention.

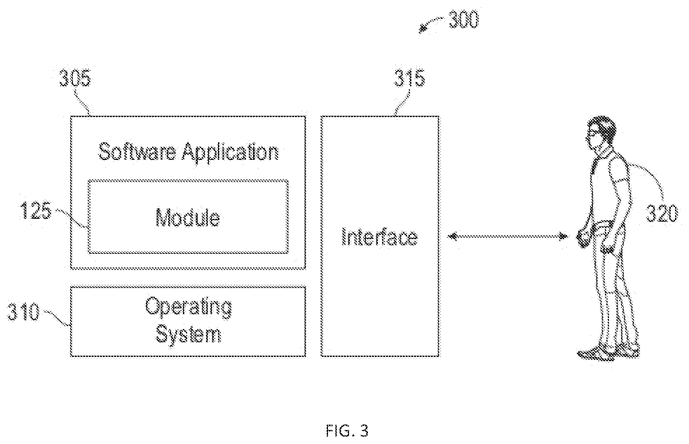

FIG. 3 illustrates a software system 300, which may be employed for directing the operation of the data-processing systems such as computer system 100 depicted in FIG. 1. Software application 305, may be stored in memory 104, on removable storage 112, or on non-removable storage 114 shown in FIG. 1, and generally includes and/or is associated with a kernel or operating system 310 and a shell or interface 315. One or more application programs, such as module(s) or node(s) 125, may be “loaded” (i.e., transferred from removable storage 114 into the memory 104) for execution by the data-processing system 100. The data-processing system 100 can receive user commands and data through user interface 315, which can include input 116 and output 118, accessible by a user 320. These inputs may then be acted upon by the computer system 100 in accordance with instructions from operating system 310 and/or software application 305 and any software module(s) 125 thereof.

Generally, program modules (e.g., module 125) can include, but are not limited to, routines, subroutines, software applications, programs, objects, components, data structures, etc., that perform particular tasks or implement particular abstract data types and instructions. Moreover, those skilled in the art will appreciate that elements of the disclosed methods and systems may be practiced with other computer system configurations such as, for example, hand-held devices, mobile phones, smart phones, tablet devices multi-processor systems, microcontrollers, printers, copiers, fax machines, multi-function devices, data networks, microprocessor-based or programmable consumer electronics, networked personal computers, minicomputers, mainframe computers, servers, medical equipment, medical devices, and the like.

Note that the term “module” or “node” as utilized herein may refer to a collection of routines and data structures that perform a particular task or implements a particular abstract data type. Modules may be composed of two parts: an interface, which lists the constants, data types, variables, and routines that can be accessed by other modules or routines; and an implementation, which is typically private (accessible only to that module), and which includes source code that actually implements the routines in the module. The term module may also simply refer to an application such as a computer program designed to assist in the performance of a specific task such as word processing, accounting, inventory management, etc., or a hardware component designed to equivalently assist in the performance of a task.

The interface 315 (e.g., a graphical user interface 130) can serve to display results, whereupon a user 320 may supply additional inputs or terminate a particular session. In some embodiments, operating system 310 and GUI 130 can be implemented in the context of a “windows” system. It can be appreciated, of course, that other types of systems are possible. For example, rather than a traditional “windows” system, other operation systems such as, for example, a real-time operating system (RTOS) more commonly employed in wireless systems may also be employed with respect to operating system 310 and interface 315. The software application 305 can include, for example, module(s) 125, which can include instructions for carrying out steps or logical operations such as those shown and described herein.

The following description is presented with respect to embodiments of the present invention, which can be embodied in the context of, or require the use of, a data-processing system such as computer system 100, in conjunction with program module 125, and data-processing system 200 and network 202 depicted in FIGS. 1-3. The present invention, however, is not limited to any particular application or any particular environment. Instead, those skilled in the art will find that the system and method of the present invention may be advantageously applied to a variety of system and application software including database management systems, word processors, and the like. Moreover, the present invention may be embodied on a variety of different platforms including Windows, Macintosh, UNIX, LINUX, Android, Arduino, LabView and the like. Therefore, the descriptions of the exemplary embodiments, which follow, are for purposes of illustration and not considered a limitation.

The embodiments disclosed herein are directed to methods and systems for nanoflow liquid chromatography making use of Mesoporous Graphitized Carbon (MGC) packed columns.

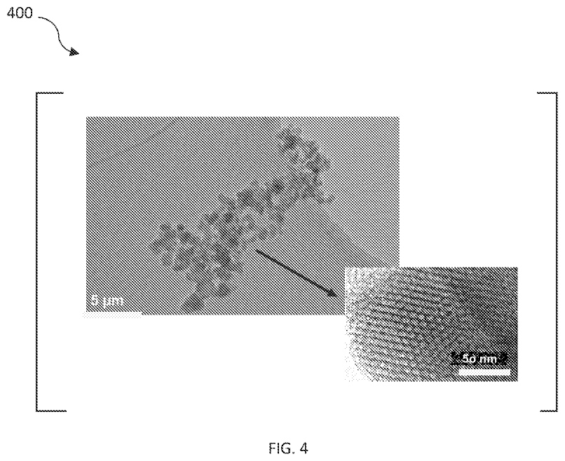

Mesoporous graphitized carbon (MGC) is a carbon based particle, which can be used as a packing material in a chromatographic column as disclosed herein. MGC can have a small particle size (less than 500 nm) and pore size (pore diameters of approximately 64 Angstroms) which provides a large surface area to interact with molecules in a chromatographic column. FIG. 4 illustrates a Transmission Electron Microscopy (TEM) image 400 of MGC particles in accordance with the disclosed embodiments.

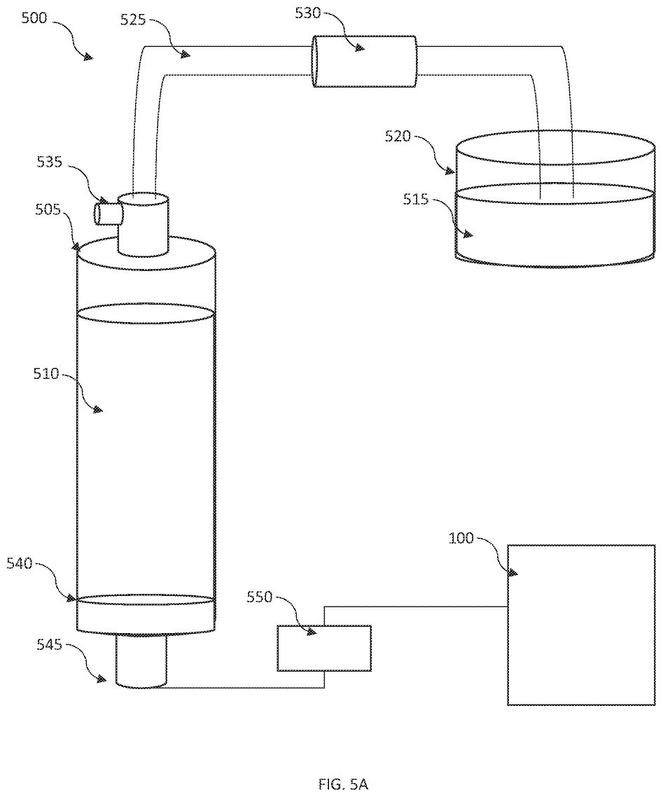

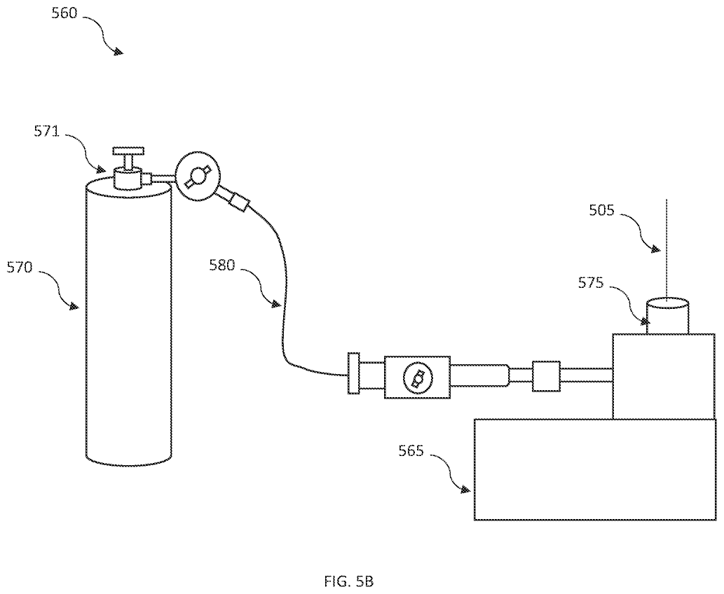

FIG. 5A illustrates a system 500 for chromatography in accordance with the disclosed embodiments. The system can comprise a column 505. The column 505 can comprise a fused silica capillary and can withstand pressures up to and including 50 MP. In other embodiments, the column can comprise a pulled capillary emitter column. The length of the column can be configured to be 10 mm, with an internal diameter of 150 micrometers or less. Note, such dimensions are optimized to achieve isomeric separation of permethylated glycans as disclosed herein, but in other embodiments, other dimensions may be appropriate for other applications.

The system can further comprise a stationary phase 510 comprising MGC. Using MGC as the stationary phase is advantageous because of its relatively small particle size and large surface area. MGC is therefore efficient for isomeric separation of permethylated glycans and glycopeptides. Frit 540 can also be configured in the column 505 at the packed end. Frit 540 can comprise potassium silicate frit, or other such frit.

The mobile phase 515 can be stored in a storage reservoir 520. The mobile phase 515 is pumped via conduit 525, with a pump 530 which can comprise a high pressure pump. Conduit 525 can be fitted with a sample injector port 535 in certain embodiments.

The system 500 can further include an output port 545 that is coupled to a detection apparatus 550. In certain embodiments, the detection apparatus 550 can comprise a mass spectrometer. The detection system can further be connected to a computer system 100, as illustrated in FIGS. 1-3. The results of the sample test can be provided in a GUI associated with the computer system 100. The detector is configured to identify constituent elements as a function of time which can then be used for identification of the constituent molecules and their relative abundance

The liquid chromatography (LC) system 500 with packed MGC column 505, enables the isomeric separation of, for example, permethylated glycans. The data is generated as a function of time. The separated isomeric glycans can be introduced into a mass spectrometer (MS) which analyses them based on their mass to charge ratio (m/z). The results from LC-MS can be made visible on a GUI associated with the computer system 100.

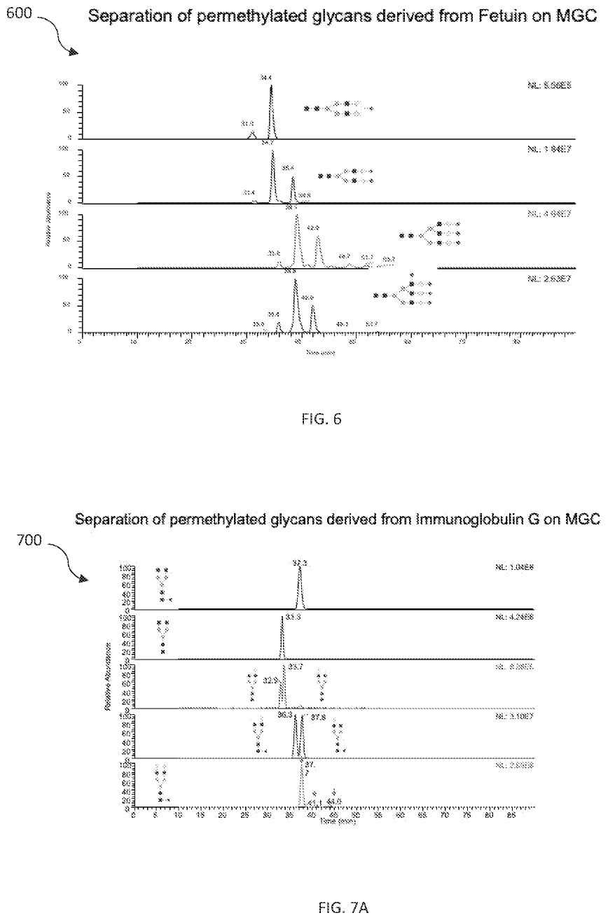

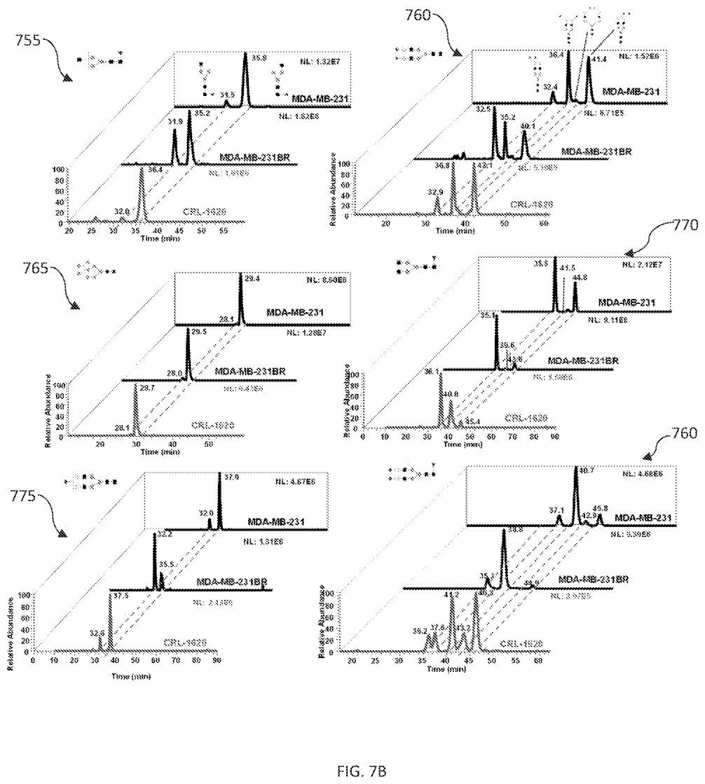

Sample output data is provided in FIG. 6, FIG. 7A, and FIG. 7B. FIG. 6 illustrates a chart 600 of chromatograms generated using the disclosed system, with isomeric separation of reduced and permethylated glycans derived from model glycoprotein fetuin. FIG. 7A illustrates a chart 700 generated from isomeric separation of reduced and permethylated glycans derived from immunoglobulin G, using the disclosed system.

The disclosed system can be used for efficient isomeric separation of permethylated glycans using breast cancer cell lines (MDA-MB231, MDA-MB-231BR) and a brain cancer cell line (CRL-1620) as well. In FIG. 7B separation of isomeric permethylated glycans derived from MDA-MB231, MDA-MB-231BR and CRL-1620, using a system 500 comprising an MGC packed column. Chart 755 in FIG. 7B shows separation of HexNAc3Hex3DeoxyHex1. Chart 760 in FIG. 7B shows separation of HexNAc4Hex5NeuAc2. Chart 765 in FIG. 7B shows separation of HexNAc2Hex8. Chart 770 in FIG. 7B shows separation of HexNAc4Hex3DeoxyHex1. Chart 775 in FIG. 7B shows separation of HexNAc4Hex5NeuAc1. Chart 780 in FIG. 7B shows separation of HexNAc4Hex5DeoxyHex1NeuAc2.

FIG. 5B illustrates a column packing assembly 560 in accordance with the disclosed embodiments. In order to pack the column 505, the stationary phase 510, can be suspended in solvent, and is then introduced into the column 505 with the help of a pressure cell 565 that is further connected to a pressurized gas (nitrogen or helium) cylinder 570. The connection can include valve and pressure fittings 571 which allow gas to be provided to the pressure cell 565.

In certain embodiments, the solvent can comprise tetrahydrofuran (THF) and can be used to dissolve MGC. The solvent-MGC suspensions can be stored in a vial 575. This vial 575 can be configured to engaged pressure cell 565. The pressure cell 565 is connected to a pressurized nitrogen or helium gas cylinder 570 with tubing 580. The Non-fritted end of capillary for packing the column is placed inside the solvent vial 575. For packing the column, a pressure of 800-1000 psi is applied by the pressure cell 565. Thus, MGC suspended in the solvent flows from the non-fritted end to the fritted end of the column 505, where stationary phase packing takes place. The fritted end of the capillary need not be connected to anything.

In an exemplary embodiment, MGC can be suspended in tetrahydrofuran and can be used to pack a 10 mm column. The column can comprise a flexible fuse silica capillary with an internal diameter of 10 micrometers. A potassium silicate frit can be inserted to close the packed end. The MGC packed column provides better resolution as compared to PGC packed columns. High backpressure resulting from the smaller particle size is being controlled by reducing the packing length and increasing the diameter of the column as compared to PGC.

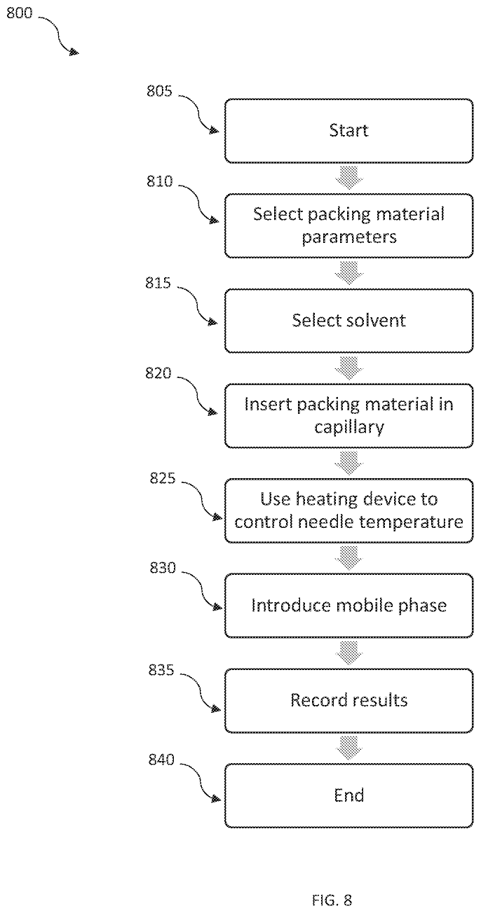

FIG. 8 illustrates steps associated with a method 800 for packing and using an MGC packed column in accordance with the disclosed embodiments. The methods starts at 805. First, the packing material parameters can be selected as shown at 810. In certain embodiments, MGC can be selected with a particle size of less than 500 nm and pore size of approximately 64 Angstroms (other sizes can also be used). The solvent can be selected at step 815 to be Tetrahydrofuran, and the capillary can be selected to be a flexible fused silica capillary with an internal diameter of 105 micrometers (or other desired internal diameter).

Next at step 820, a pressure injection cell can be used to pull the packing material in the capillary. The packed end of the capillary can have a potassium silicate frit inserted which serves as a platform for packing material to settle and prevents the packing material from exiting the column. MGC suspended in THF is then introduced into the capillary with the help of the pressure cell.

The packed MGC column or pulled capillary nanospray emitter column can be used with the system to obtain isomeric separation of, for example, permethylated glycans at 75 degrees Celsius. A heating device can be used at step 825 to control the temperature in the case where a pulled capillary nanospray emitter column is used. The heater is required for the pulled capillary nanospray emitter because this column is placed inside the electrospray ionization (ESI) source of the mass spectrometer, which does not have temperature control. This heater may not be required in the case where an MGC capillary column is used because such a column can reside in an LC column compartment which may be equipped with the ability to control the temperature. Optimal separation is provided at approximately 75 degrees Celsius. Finally, at step 830, permethylated glycan samples and the mobile phase can be driven through the column and at step 835 results can be recorded. The method ends at 840.

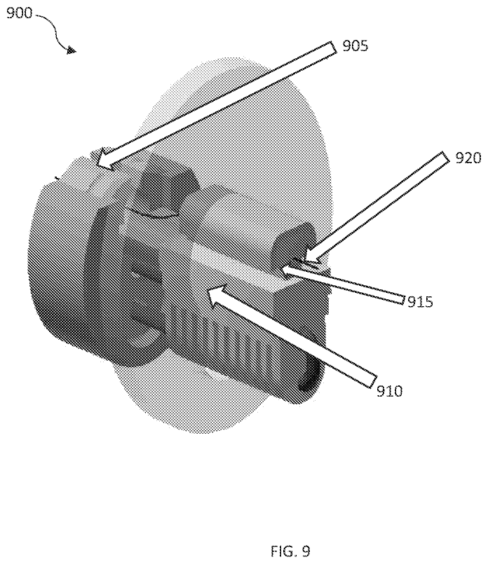

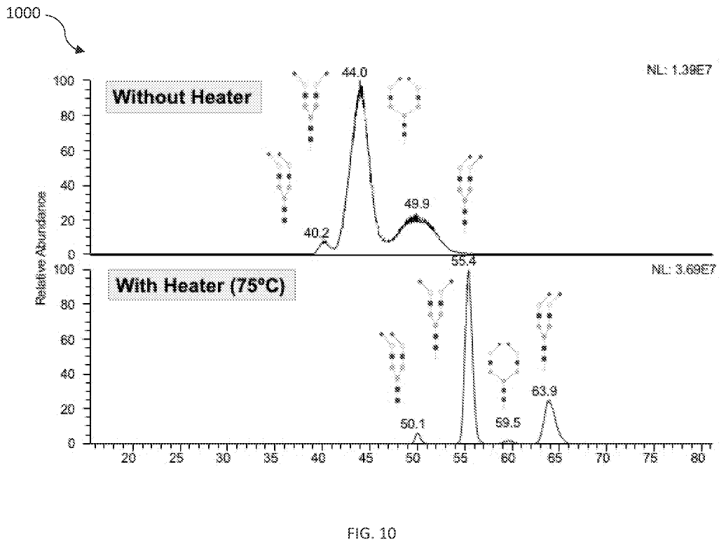

FIG. 9 illustrates a heating device 900 for a pulled capillary nanospray emitter column in accordance with the disclosed embodiments. The heating device 900 includes a needle assembly 905 (or pulled capillary nanospray emitter column assembly), attached to a cold plate 910. A hot plate 915 is configured above, and in thermal communication with, the cold plate 910 with the pulled tip capillary 920 extending therefrom. The heating device 900 is used for controlling the temperature of the needle. Since, optimum separation is achieved at 75° C., and the needle is used outside the LC compartment. The heating device 900 can be configured inside the ESI source, where the pulled capillary nanospray emitter column operates. The heating device 900 can be controlled by software present on the computer system 100. FIG. 10 illustrates a chart 1000 of relative measurements with and without the heater.

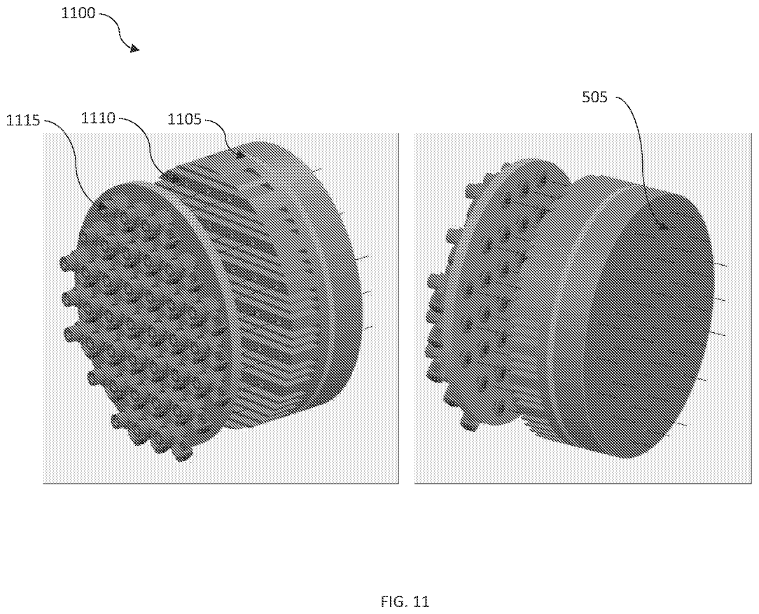

FIG. 11 illustrates a multiarray of heated injectors 1100 for an MGC needle column in accordance with the disclosed embodiments. This system is designed to reduce the time involved in the process of reassembling different needle columns. The multiarray device 1100 includes a stage 1105 which has slots 1110 for housing multiple MGC packed pulled capillary nanospray emitter columns 505. Thus, multiple columns can be installed on the microarray 1100. Replacing the column every time a new one is required is time consuming and may require removal and re-installation of the heating device. As such, the use of this microarray 1100 which can accommodate multiple packed nanospray emitters offers a significant time advantage. If an MGC packed emitter needs to be replaced with a new one, the capillary connection from LC can be made with the corresponding T-junction 1115 instead of removing the whole installation for replacement.

As such, in an exemplary embodiment, a chromatographic system can include a column packed with MGC. The MGC serves as the stationary phase and facilitates the efficient isomeric separation of compounds such as permethylated glycans.

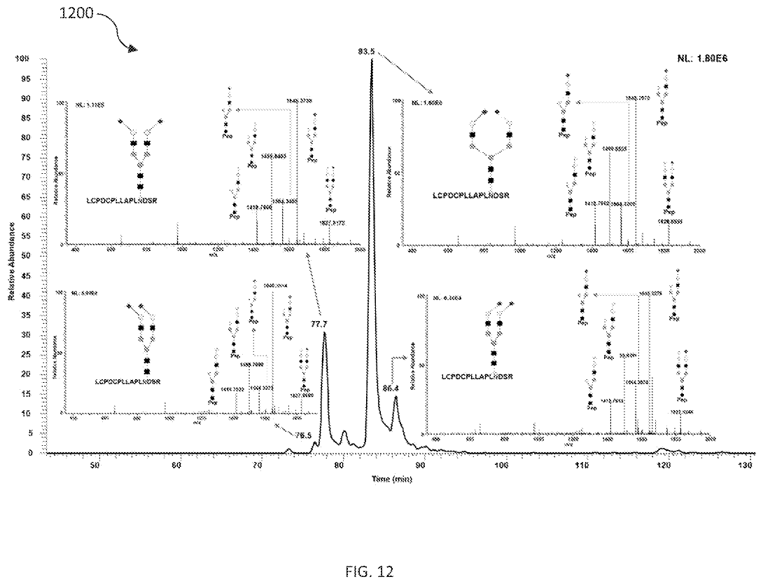

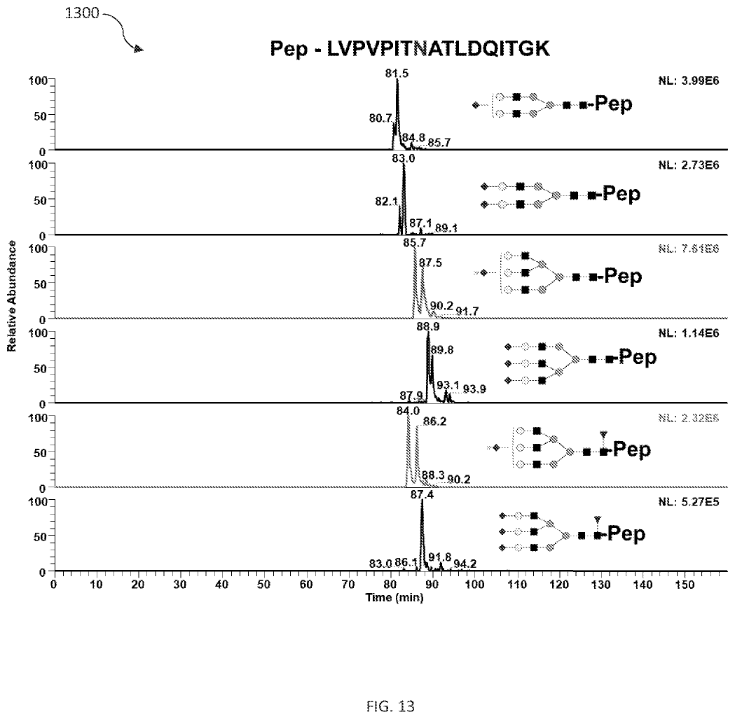

MGC columns are more stable as well and more reproducible as compared to prior stationary phase materials such as PGC. The MGC can be suspended in tetrahydrofuran and can be used to pack 10 mm of column length. The column can comprise a flexible fused silica capillary with an internal diameter of 150 μm. A potassium silicate frit can be inserted to close the packed end. Permethylated glycans derived from human blood serum as well as breast cancer and brain cancer cell lines were tested on MGC to examine the biological applicability of the column. The stationary phase can also be packed in the pulled tip capillary nanospray emitter which can be used as a column to decrease the dead volume and improve peak resolution. Moreover, in this unprecedented development, efficient isomeric separation of permethylated glycans has been achieved with only 10 mm long column. The column is an economical replacement (can be prepared in less than $10) to the commercial PGC column which was priced over $1000. FIG. 12 illustrates a chart 1200 showing an MS/MS spectrum of isomeric A2G2S2 glycopeptide from bovine fetuin separated on an MGC column in accordance with the disclosed embodiments. FIG. 13 illustrates a chromatogram 1300 showing the isomeric separation of glycopeptides derived from alpha-1 acid glycoprotein on an MGC column in accordance with the disclosed embodiments.

Based on the foregoing, it can be appreciated that a number of embodiments, preferred and alternative, are disclosed herein. For example, in an embodiment, a chromatography system comprises a column, a stationary phase comprising mesoporous graphitized carbon, and frit inserted in the column.

In an embodiment the chromatography system further comprises a fused silica capillary. In an embodiment, the column comprises a pulled capillary nanospray emitter column.

In an embodiment, the mesoporous graphitized carbon has a size of less than 500 nm and a pore size of 64 Angstroms. In an embodiment, the column further comprises a packed length of 10 mm. In an embodiment, the system is configured for efficient isomeric separation.

In an embodiment the chromatography system further comprises the stationary phase is inserted into the column as a solvent-MGC suspension. In an embodiment, the solvent-MGC suspension comprises Tetrahydrofuran and the mesoporous graphitized carbon.

In another embodiment, a chromatography method comprises packing a column with a stationary phase comprising mesoporous graphitized carbon, introducing a mobile phase in the column, separating constituent elements in the mobile phase, and identifying the constituent elements in the mobile phase.

In an embodiment of the method, the column comprises one of: a fused silica capillary and a pulled capillary nanospray emitter column. In an embodiment, the method further comprises selecting the mesoporous graphitized carbon to have a size of less than 500 nm and a pore size of 64 Angstroms.

In an embodiment packing the column with a stationary phase further comprises inserting a frit in the column. In an embodiment, packing the column with a stationary phase further comprises selecting a solvent and creating a suspension of the solvent and mesoporous graphitized carbon. In an embodiment, the suspension of the solvent and the mesoporous graphitized carbon comprises Tetrahydrofuran and the mesoporous graphitized carbon suspended in the Tetrahydrofuran. In an embodiment, packing the column with a stationary phase further comprises packing 10 mm of the column with the stationary phase. In an embodiment, separating constituent elements in the mobile phase comprises isomeric separation of the constituent elements.

In an embodiment of the method, the mobile phase comprises one of HexNAc3Hex3DeoxyHex1, HexNAc4Hex5NeuAc2, HexNAc2Hex8, HexNAc4Hex3DeoxyHex1, HexNAc4Hex5NeuAc1, and HexNAc4Hex5DeoxyHex1 NeuAc2.

In an embodiment, separating constituent elements in the mobile phase comprises isomeric separation of permethylated glycans.

In another embodiment, a chromatography system comprises a column comprising a pulled capillary nanospray emitter column, a stationary phase comprising mesoporous graphitized carbon, frit inserted in the column and a heater configured for controlling a temperature of the pulled capillary nanospray emitter column. In an embodiment, the mesoporous graphitized carbon is selected to have a size of less than 500 nm and a pore size of 64 Angstroms, and the mesoporous graphitized carbon in a packed into 10 mm or less of the pulled capillary nanospray emitter column.

It will be appreciated that variations of the above-disclosed and other features and functions, or alternatives thereof, may be desirably combined into many other different systems or applications. Also, it should be appreciated that various presently unforeseen or unanticipated alternatives, modifications, variations, or improvements therein may be subsequently made by those skilled in the art which are also intended to be encompassed by the following claims.

What is claimed is:

1. A chromatography system comprising:
a column;
a stationary phase comprising mesoporous graphitized carbon; and
frit inserted in the column.

2. The chromatography system of claim 1 further comprising:
a fused silica capillary.

3. The chromatography system of claim 1 wherein the column comprises:
a pulled capillary nanospray emitter column.

4. The chromatography system of claim 1 wherein the mesoporous graphitized carbon has a size of less than 500 nm and a pore size of 64 Angstroms.

5. The chromatography system of claim 1 wherein the column further comprises:
a packed length of 10 mm.

6. The chromatography system of claim 5 wherein the system is configured for efficient isomeric separation.

7. The chromatography system of claim 1 wherein the stationary phase is inserted into the column as a solvent-MGC suspension.

8. The chromatography system of claim 7 wherein the solvent-MGC suspension comprises:
Tetrahydrofuran; and
the mesoporous graphitized carbon.

9. A chromatography method comprising:
packing a column with a stationary phase comprising mesoporous graphitized carbon;
introducing a mobile phase in the column;
separating constituent elements in the mobile phase; and
identifying the constituent elements in the mobile phase.

10. The chromatography method of claim 9 wherein the column comprises one of:
a fused silica capillary; and
a pulled capillary nanospray emitter column.

11. The chromatography method of claim 9 further comprising:
selecting the mesoporous graphitized carbon to have a size of less than 500 nm and a pore size of 64 Angstroms.

12. The chromatography method of claim 9 wherein packing the column with a stationary phase further comprises:
inserting a frit in the column.

13. The chromatography method of claim 9 wherein the packing the column with a stationary phase further comprises:
selecting a solvent; and
creating a suspension of the solvent and mesoporous graphitized carbon.

14. The chromatography method of claim 13 wherein the suspension of the solvent and the mesoporous graphitized carbon comprises:
Tetrahydrofuran; and
the mesoporous graphitized carbon suspended in the Tetrahydrofuran.

15. The chromatography method of claim 9 wherein the packing the column with a stationary phase further comprises:
packing 10 mm of the column with the stationary phase.

16. The chromatography method of claim 9 wherein separating constituent elements in the mobile phase comprises:
isomeric separation of the constituent elements.

17. The chromatography method of claim 16 wherein the mobile phase comprises one of:
HexNAc3Hex3DeoxyHex1;
HexNAc4Hex5NeuAc2;
HexNAc2Hex8;
HexNAc4Hex3DeoxyHex1;
HexNAc4Hex5NeuAc1; and
HexNAc4Hex5DeoxyHex1NeuAc2.

18. The method of claim 17 wherein separating constituent elements in the mobile phase comprises:
isomeric separation of permethylated glycans.

19. A chromatography system comprising:
a column comprising a pulled capillary nanospray emitter column;
a stationary phase comprising mesoporous graphitized carbon;
frit inserted in the column; and
a heater configured for controlling a temperature of the pulled capillary nanospray emitter column.

20. The chromatography system of claim 19 further wherein the mesoporous graphitized carbon is selected to have a size of less than 500 nm and a pore size of 64 Angstroms, and the mesoporous graphitized carbon in a packed into 10 mm or less of the pulled capillary nanospray emitter column.
